SEQUENCE LISTING (6 sequences)

SEQ ID NO: 1 - DNA, 18 bp, synthetic construct
  source 1..18: mol_type = other DNA
gacctttaggccagggag

SEQ ID NO: 2 - DNA, 18 bp, synthetic construct
  source 1..18: mol_type = other DNA
ttcgcgctcgagtcgatc

SEQ ID NO: 3 - DNA, 22 bp, synthetic construct
  source 1..22: mol_type = other DNA
atgtccagatacgttgcgtccg

SEQ ID NO: 4 - DNA, 22 bp, synthetic construct
  source 1..22: mol_type = other DNA
aggaggtatcggtggagggaac

SEQ ID NO: 5 - DNA, 25 bp, synthetic construct
  source 1..25: mol_type = other DNA
cgccctaattgaataggtgacttag

SEQ ID NO: 6 - DNA, 22 bp, synthetic construct
  source 1..22: mol_type = other DNA
taagttayactgtggagttyag

(19) United States
(12) Patent Application Publication
(10) Pub. No.: US 2024/0384361 A1
(43) Pub. Date: Nov. 21, 2024

(54) LONG-TERM SURROGATE VIRUS ASSAYS AND METHODS

(71) Applicant: REGENTS OF THE UNIVERSITY OF MINNESOTA, Minneapolis, MN (US)

(72) Inventors: Declan C. Schroeder, Minneapolis, MN (US); Cecilia Balestreri, St. Paul, MN (US); Jennifer L.G. van de Ligt, Brooklyn Park, MN (US); Gerald C. Shurson, Arden Hills, MN (US); Pedro E. Urriola Albers, Arden Hills, MN (US); Amanda M. Palowski, Forest Lake, MN (US)

(21) Appl. No.: 18/689,602

(22) PCT Filed: Sep. 15, 2022
(86) PCT No.: PCT/US2022/043622

§ 371 Date: Mar. 6, 2024

Related U.S. Application Data
(60) Provisional application No. 63/245,482, filed on Sep. 17, 2021.

(51) Int. Cl. C12Q 1/70; C12Q 1/6851

(52) U.S. Cl. CPC C12Q 1/701; C12Q 1/6851

(57) ABSTRACT

A method for monitoring the viability of a megavirus in animal feed or an animal feed ingredient generally includes inoculating the animal feed or animal feed ingredient with a surrogate virus as a proxy for the megavirus, subjecting the animal feed or animal feed ingredient to a treatment that inactivates the megavirus and the surrogate virus, subjecting the animal feed or animal feed ingredient to storage or transportation conditions for at least 14 days, and determining the viability of the surrogate virus in the animal feed or animal feed ingredient, thereby monitoring the viability of the megavirus in the animal feed or animal feed ingredient. In one or more embodiments, the megavirus is African swine fever virus (ASFV) and the surrogate virus is an ASFV surrogate virus. In some of these embodiments, the ASFV surrogate virus is a Coccolithovirus such as, for example, Emiliania huxleyi virus.

### CROSS-REFERENCE TO RELATED APPLICATION

The present application claims priority to U.S. Provisional Application No. 63/245,482, filed Sep. 17, 2021, which is incorporated herein by reference in its entirety.

### SUMMARY

This disclosure describes, in one aspect, a method for monitoring the presence or absence or the viability of a megavirus in animal feed or an animal feed ingredient. Generally, the method includes inoculating the animal feed or animal feed ingredient with a surrogate virus as a proxy for the megavirus, subjecting the animal feed or animal feed ingredient to a treatment that inactivates the megavirus and the surrogate virus, subjecting the animal feed or animal feed ingredient to storage or transportation conditions for at least 14 days, and determining the presence or absence or the viability of the surrogate virus in the animal feed or animal feed ingredient, thereby monitoring the presence or absence or the viability of the megavirus in the animal feed or animal feed ingredient.

In one or more embodiments, the megavirus is African swine fever virus (ASFV) and the surrogate virus is an ASFV surrogate virus. In some of these embodiments, the ASFV surrogate virus is a Coccolithovirus such as, for example, Emiliania huxleyi virus.

In one or more embodiments, the treatment that inactivates infectious ASFV and the ASFV surrogate virus includes exposure to a temperature of at least 65° C. for at least one minute, exposure to a temperature of at least 85° C. for at least one second, exposure to citric acid, or exposure to increased salinity.

In one or more embodiments, the animal feed or animal feed ingredient is subjected to storage or transportation conditions for at least 120 days. In one or more of these embodiments, the animal feed or animal feed ingredient is subjected to storage or transportation conditions for at least 180 days.

In one or more embodiments, the conditions of storage or transportation include temperatures up to and including 100° C.

In one or more embodiments, the method further includes determining that the animal feed or animal feed ingredient with no detectable viable surrogate virus is safe for livestock.

In one or more embodiments, the method further includes identifying a treatment that results in no detectable viable surrogate virus as effective to permanently inactivate the megavirus

In one or more embodiments, the method further includes comparing the thermal stability of the virus surrogate and megavirus to a virus known to have low thermal stability (e.g. Porcine reproductive and respiratory syndrome virus (PRRSV)) across a range of temperatures.)

In another aspect, this disclosure describes a method for monitoring the presence or absence or the viability of a megavirus in an animal feed or an animal feed ingredient. Generally, the method includes inoculating the animal product with a surrogate virus as a proxy for the megavirus, subjecting the animal product to a treatment that inactivates the megavirus and the surrogate virus, subjecting the animal feed or animal feed ingredient to storage or transportation conditions for at least 14 days, and determining the presence or absence of the viable surrogate virus in the animal product, thereby monitoring the presence or absence of the viable megavirus in the animal product.

In one or more embodiments, the animal feed or animal feed ingredient is subjected to storage or transportation conditions for at least 120 days. In some of these embodiments, the animal feed or animal feed ingredient is subjected to storage or transportation conditions for at least 180 days.

In one or more embodiments, the conditions of storage or transportation include temperatures up to and including 100° C.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Map of the route traveled by a commercial truck carrying test feed ingredients inoculated with Emiliania huxleyi virus (EhV-86).

FIG. 2. Standard curve obtained for EhV-86 PCR assays. Number of copies of DNA in the dilution series (one microliter (μL) used per dilution) against Ct (cycle threshold) values as obtained by real time PCR, with each dot representing three technical replicates. Bars=standard deviation from the mean.

FIG. 3. EhV-86 quantity in conventional soybean meal (SBMC) as detected by standard qPCR (S) and viability qPCR (V) using EhV-86 eluant without extracting DNA and after extracting DNA. Bar=standard deviation from the mean.

FIG. 4. Boxplot of EhV-86 quantity (virus/μL) in complete feed (CF), conventional soybean meal (SBMC), and organic soybean meal (SBMO) with viable (V) and non-viable or standard (S) DNA presence on day 0 post infection (DPI_0) and after 23-day commercial truck journey (DPI_23).

FIG. 5. EhV-86 quantity (virus/g) relative to Ct values in complete feed (CF), conventional soybean meal (SBMC), and organic soybean meal (SBMO) with viable (V) and non-viable or standard (S) DNA presence after a 23-day commercial truck journey.

FIG. 6. Graphical representation of virions and putative evolutionary relationships of nucleocytoplasmic large DNA viruses (NCLDVs). The ancestral NCLDV diverged into two superclades, PAM (Phycodnaviridae-Asfarviridae-Megavirus) and MAPI (Marseilleviridae-Ascoviridae-Pithovirus-Iridoviridae), with poxviruses forming a distinct monophyletic lineage. This topology is based on the inference trees produced by comparing a single core gene such the RNA polymerase or multiple core proteins. Capsid (red), protein core (navy blue dots), inner-membrane (blue), outer-membrane (yellow), and envelope (green) are all illustrated when applicable. The names of viruses that are known to possess an envelope are highlighted by dashed boxes. The two algae-infecting PAM members, Chlorovirus and Coccolithovirus, within the family Phycodnaviridae are connected by green shaded oval, whereas the giant amoeba-infecting members are connected by a blue shaded oval. The virus representations are not to scale and were created using BIORENDER (Biorender.com).

FIG. 7. Infectivity of EhV-86 after temperature treatment. A log₁₀ E. huxleyi cell density (cells/mL) plot indicating the capacity of temperature treated EhV-86 to induce E. huxleyi lysis, with subsequent change in cell density in infected cultures eight days post-virus inoculation in bioassays. EhV-86 was treated for short time-intervals ranging from 30 seconds to 300 seconds and at temperatures from 4° C. to 60° C. Bars show the SD of the mean of five replicates.

FIG. 8. EhV-86 particles observed using confocal microscopy after temperature treatment. Each 200 nanometer (nm) dot (green) corresponds to one virus particle. All images are merged composites of both blue (DNA stain, DAPI) and red fluorescence (lipid stain, FM 1-43) captured using excitation settings of 405 nm and 488 nm, respectively. For ease of visualisation, the DAPI fluorescence images are shown in green. The images show DNA-containing viral particles after 20-minute treatment at (A) 4° C., (B) 60° C., (C) 80° C. and (D) 100° C. White arrows in panel A indicate example virus particles.

FIG. 9. Inactivation kinetics of EhV-86. (A) One million initial EhV-86 particles were treated at temperatures ranging from 4° C. to 100° C. for 20 minutes. Mean virus counts (SD from three biological replicates shown) as measured by standard(S) and viability (V) qPCR were determined for each of three temperature treatments. (B) Temperature and time inactivation kinetics as determined from viability qPCR for EhV-86 treated at 60° C., 80° C., and 100° C. for 10 minutes, 20 minutes, 40 minutes, or 60 minutes. A Weibull model was fitted to the data with R² values of 0.93, 0.97 and 0.95, to the 60° C., 80° C., and 100° C. data sets, respectively.

FIG. 10. Changes in EhV particle properties at elevated temperatures using Flow cytometry (FC). (A-D) FITC vs SSC plots of EhV particles treated at 4° C., 48° C., 60° C., and 80° C. for 20 minutes. Location of infectious (black), VNIP (blue), and structurally damaged EhV-86 (red) are indicated by arrows. SPHEROTECH beads (1.32 um) are run as a size reference in every FC run. The lower panel is a schematic representation of the EhV virion structure degrading at different temperatures at each exposure. The virus representations were created with BIORENDER (Biorender.com).

FIG. 11. Comparing the heat inactivation kinetics of EhV-86 (A), ASFV (B) and PRRSV (C). (A) One million initial EhV-86 particles were treated at temperatures ranging from 4° C. to 100° C. for 20 mins. Mean virus counts (SD from three biological replicates shown) as measured by standard(S) and viability (V) qPCR were determined for each of three temperature treatments. (B) One hundred thousand initial ASFV particles were treated at temperatures ranging from 4° C. to 100° C. for 20 mins. Mean virus counts (SD from three biological replicates shown) as measured by standard(S) and viability (V) qPCR were determined for each of three temperature treatments. (C) One thousand median tissue culture infectious dose (TCID₅₀) equivalent infectious units of PRRSV particles treated at temperatures ranging from 4° C. to 100° C. for 20 mins. Mean virus counts (SD from three biological replicates shown) as measured by standard(S) and viability (V) qPCR were determined for each of three temperature treatments.

### DETAILED DESCRIPTION OF ILLUSTRATIVE EMBODIMENTS

African swine fever virus (ASFV) is a nucleocytoplasmic large DNA virus (NCLDV) that is stable in a variety of environments, including animal feed ingredients as shown in previous laboratory experiments and simulations. Emiliania huxleyi virus (EhV) is the only known species within the Coccolithovirus genus of NCLDV. It has a restricted host range limited to the marine algae Emiliania huxleyi. This algal NCLDV has many similar morphological and physical characteristics to ASFV thereby making it a safe surrogate, with results that are applicable to ASFV, as described in International Patent Application No. PCT/US2020/039072, which published as International Publication No. WO 2020/263788A1.

This disclosure describes an assay for monitoring the presence or absence of a megavirus in animal feed, an animal feed ingredient, or an animal product. This assay may additionally or alternatively measure the viability of a megavirus in animal feed, an animal feed ingredient, or an animal product. The assay can be used as a surveillance tool to determine whether a particular unit of animal feed, animal feed ingredient, or animal product is free of megavirus and therefore safe to provide to livestock. The assay also can be used to determine whether a particular virus-inactivating treatment is effective for inactivating a megavirus for a predetermined period of time such as, for example, a period of time that corresponds to real-world commercial periods of transportation or storage.

The assay described herein is a risk-free in situ non-animal (RISNA) megavirus surrogate model assay that can be performed to reduce the risk of transmitting megaviruses in animal feed or animal feed ingredients in the supply chain. The data provided herein demonstrate that the surrogate virus EhV-86 remains viable in animal feed matrices throughout typical commercial transportation conditions, indicating that the assays described herein have direct commercial applicability surveilling animal feed or animal feed ingredients and/or evaluating the safety of animal feed or animal feed ingredients after transportation.

In one or more exemplary embodiments, the megavirus being monitored using the surrogate assay is African swine fever virus (ASFV). While described herein in the context of an exemplary embodiment in which megavirus being monitored is ASFV, the assay method described herein can be used to monitor other megaviruses. Exemplary alternative megaviruses that can be monitored using the assay include, for example, Members of the families Poxviridae, Mimiviridae, Marseilleviridae, Iridoviridae, and other double-stranded DNA (dsDNA) viruses—e.g., members of the family Herpesviridae. While poxviruses have somewhat different morphology, the surrogate assay may still be used to effectively monitor dsDNA genome degradation in the poxviruses.

ASFV is a complex enveloped virus that belongs to a group of megaviruses that replicate completely or partly in the cytoplasm of eukaryotic cells. African swine fever is a highly contagious disease manifesting clinical symptoms of hemorrhagic fever caused by ASFV and leading to almost 100% mortality in domestic pigs. Infected pigs typically die within one month after the first clinical signs appear.

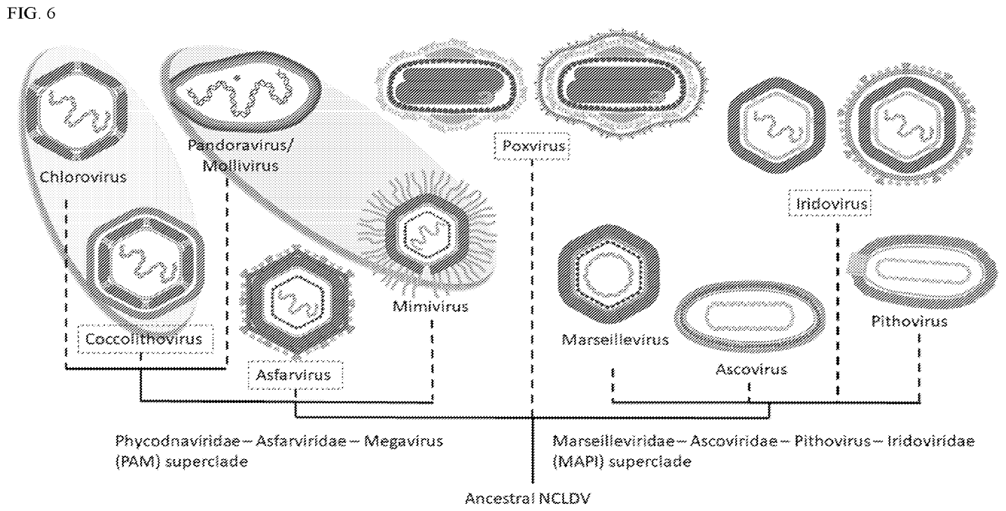

African swine fever virus, family Asfarviridae belongs to a group of viruses known as nucleocytoplasmic large dsDNA viruses (NCLDVs) or megaviruses that replicate completely or partly in the cytoplasm of eukaryotic cells. Megaviruses are a diverse group of viruses that include Ascoviridae, Asfarviridae, Iridoviridae, Marseilleviridae, Mimiviridae, Phycodnaviridae, and Poxviridae, plus several lineages of unclassified viruses, such as pithoviruses, pandoraviruses, molliviruses, and faustoviruses (FIG. 6). Megaviruses are united by sharing at least five conserved core genes. ASFV shares a similar icosahedral morphology with members of the family Iridoviridae and does not resemble the brick-shaped poxviruses (Poxviridae). ASFV does not fit well into any of the animal-related megavirus families—i.e., megaviruses that infect animals.

ASFV is the only member of the family Asfarviridae, but it shares many similarities with a non-animal member of the megaviruses, Emiliania huxleyi virus (EhV), a Coccolithovirus (FIG. 6). The similarities between ASFV and EhV make EhV an effective surrogate for ASFV in embodiments of the assay that are designed to monitor ASFV. In contrast, Porcine reproductive and respiratory syndrome virus (PRRSV) is a single positive-stranded RNA virus of the genus Asfivirus, which is also enveloped virus but lacks the inner membrane and stability properties of that of a dsDNA genome.

Thus, in one or more embodiments in which the megavirus being monitored using the surrogate assay is African swine fever virus (ASFV), EhV can be the surrogate virus used in the surrogate assay. While described herein in the context of an exemplary embodiment in which EhV is a surrogate virus for ASFV, the assay methods described herein can be any suitable virus that is a suitable proxy for the virus being monitored. Exemplary alternative surrogate viruses for ASFV include, for example, phaeoviruses, algal viruses of the genus Phaeovirus that are morphologically and genetically similar to EhV. In the examples provided, the EhV strain EhV-86 was used. EhV-86 is commonly used strain of Coccolithovirus, however, the assays described herein can be adapted to use different strains or isolates.

Generally, an alternative surrogate virus is morphologically similar to the virus for which it is a surrogate. Also, the surrogate virus and the virus being monitored should react similarly to virus-inactivating treatments. In some embodiments, the surrogate virus does not infect humans or animals. In some embodiments, the surrogate virus will not produce any substance (e.g., a toxin) that causes an undesirable biological effect or interacts in a deleterious manner with the livestock to which the animal feed is to be provided or with humans. In one or more embodiments, it may be advantageous to measure the inactivation of an additional virus unrelated to either the surrogate virus or the megavirus. Particularly in embodiments wherein the megavirus is known to be adaptable to a wide variety of conditions, it can be useful to include a less stable virus as a control. In one or more of the embodiments described herein, viability of an unrelated virus is measured in parallel with the surrogate virus and the megavirus. Viability data of the unrelated virus may provide more information about whether the surrogate virus accurately represents the behavior of the megavirus of interest.

There is significant variability in the survival of, for example, porcine viruses in feed ingredients and complete feed. In some feed ingredients, the viruses survive for extended periods of time (e.g., soybean meal), but in others (e.g., complete feed), they are rapidly inactivated or survive for a short time. This variability implies that the feed characteristics (e.g., processing temperature, water activity, pH, salinity) affect virus survival, leading to some feed ingredients being at greater risk of viral transmission. However, to date, this information has been collected by testing survival in vitro. Due to the high risk associated with these highly contagious and infectious viruses, no real-world in situ data exists. The current in vitro empirical approach of determining at risk feed testing combinations of viruses and feed is unsustainable due to the magnitude of virus and feed ingredient combinations. The surrogate virus assays described herein provide a platform for evaluating practical solutions feed mills can use to ameliorate ‘risk’ of transmitting ASFV and other megaviruses.

The surrogate virus assays described herein can be used as a surveillance assay, providing real-world in situ data on the effectiveness of treatments in reducing—even eliminating—a megavirus from complete feed produced at scale. The assay may be employed post-production in a feed mill. The assay also may be employed before feed is stored or transported to ensure that the feed being distributed is safe for livestock consumption. In addition, or alternatively, the assay may be performed after storage and/or transport to ensure that the feed being received is safe for livestock consumption.

The surrogate virus assay described herein can also be used to develop new mitigation techniques. For example, the assays described herein can be used to evaluate and screen anti-microbial, anti-viral, or related mitigation compounds, strategies, and protocols.

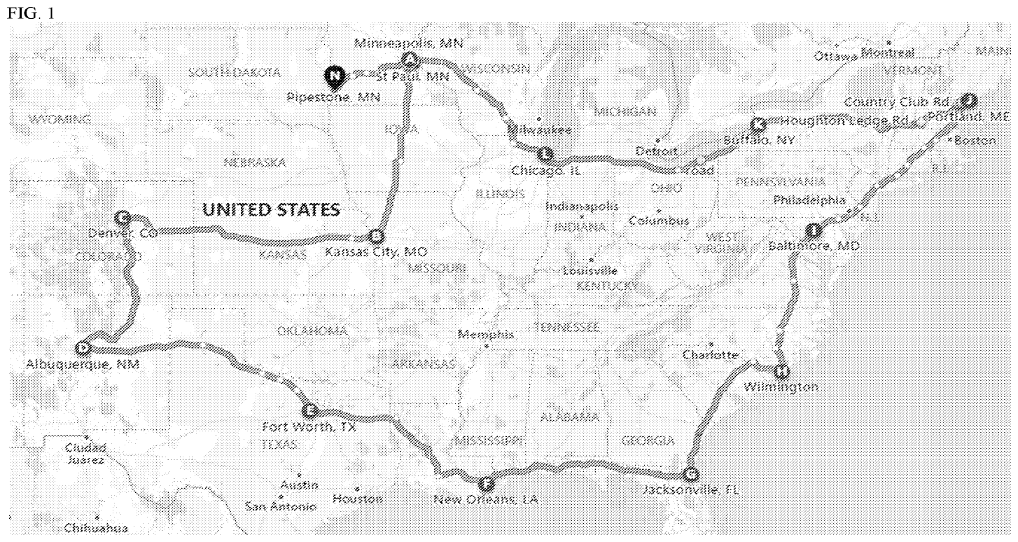

This disclosure describes an exemplary use of EhV as a surrogate for ASFV in various animal feed matrices in real-world conditions. Specifically, conventional soybean meal, organic soybean meal, and complete feed were inoculated with EhV-86, subjected to a 23-day transportation period, then tested for viability using viability qPCR (V-qPCR). Conventional soybean meal (SBMC), organic soybean meal (SBMO), and swine complete feed (CF) matrices were inoculated with EhV-86 at a concentration of 6.6×10⁷ virus g⁻¹. The inoculated feed matrices were then transported in the trailer of a commercial transport vehicle for 23 days across 10,183 km covering 29 states in various regions of the United States (FIG. 1). Upon return, samples were evaluated for virus presence and viability using a previously validated v-qPCR method. EhV-86 was detected in all matrices and no degradation in EhV-86 viability was observed after the 23-day transportation event. Additionally, sampling sensitivity rather than virus degradation best explains the variation of virus quantity observed after 23-day transport. These results demonstrate for the first time that ASFV-like NCLDVs can retain viability in swine feed matrices during long-term transport across the continental United States. While described below in the context of periods of transportation, the data further support real-world storage conditions, regardless of whether the animal feed or animal feed ingredient are actually transported.

### qPCR Standards

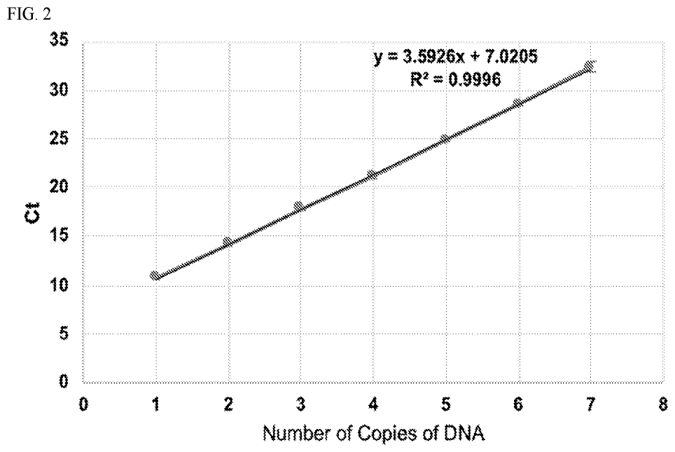

A 10-fold serial dilution of the EhV-86 MCP qPCR amplicon was used to create a standard curve (y=3.5926x+7.0205) to convert the Ct values from the qPCR assay to EhV-86 genomic equivalents (FIG. 2). The standard curve had an R² value of 0.9996 indicating high accuracy of prediction (Bustin, S. and Hugget, J., 2017, Biomol. Detect. Quantif. 14:19-28). The EhV-86 MCP qPCR assay had a quantifiable range of 10 to 10 million EhV-86 genomes per reaction.

### Virus Extraction Efficiency of Conventional Soybean Meal

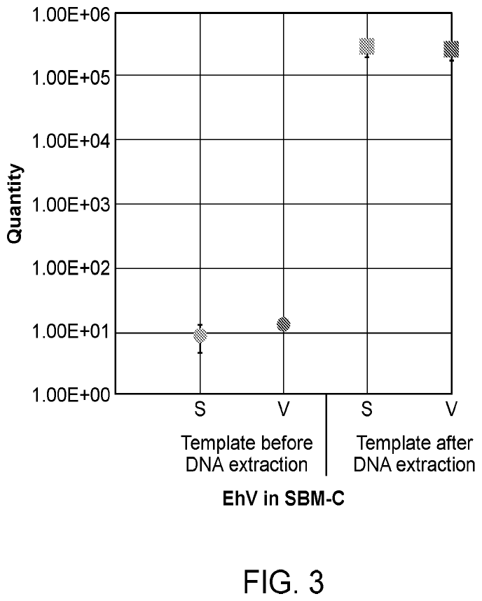

Before performing multiple extractions from feed samples, the EhV-86 elution protocol was evaluated using a conventional soybean meal sample (SMBC). The protocol was also tested to determine whether the eluants could be used directly as templates in the qPCR, or if EhV-86 DNA needed to be extracted from the final eluant. 8.53×10⁰ EhV-86 per microliter (μL) (4.26×10² EhV-86/g, 0.01% recovery; ±4.0 SD, Ct 40.4) were detected from the eluant using standard (S) qPCR; 1.26×10¹ EhV-86/μL (6.28×10² EhV-86/g; ±0 SD, Ct 38.2) were detected from the eluant using V-qPCR (FIG. 3). In contrast, performing DNA extraction on the eluant significantly increased the amount of virus that was detected via standard (22.8%) and V-qPCR (19.9%; p=0.012; FIG. 3). Comparing the detection rates of both qPCR assays, similar or identical recovery percentages were obtained (p=0.81), suggesting that EhV-86 is stable in SBMC over the five-minute assay incubation period. Moreover, the elution efficiency of EhV-86 from SBMC was about 20%, indicating that about 80% of the virus remained attached or associated to the feed matrix is some way.

### Virus DNA Detected on Feed Matrices Transported Across the United States

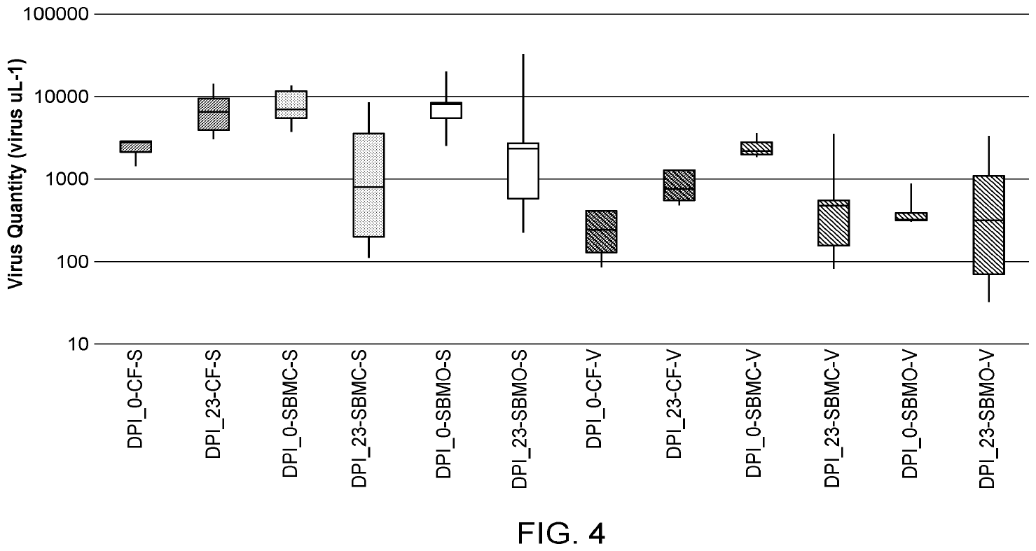

On day 0, 30 g of each type of feed sample were inoculated with 2 milliliters (mL) of EhV-86 at a concentration of 1×10⁹ viruses/mL, which resulted in an initial virus load of 6.6×10⁷ viruses/g of feed matrix. For the inoculated CF samples, an average of 2.36×10³ EhV-86/μL of eluant (Ct 24.9) or 1.18×10⁵ EhV-86/g of CF was recovered on d 0, representing only a 0.2% average recovery rate. In inoculated SBMC samples, 6.83×10³ EhV-86/μL (Ct 23.3) or 3.41×10⁵ EhV-86/g were recovered. In inoculated SBMO samples, 8.61×10³ EhV-86/μL (Ct 23.2) or 4.31×10⁵ EhV-86/g were recovered. The average recovery rate for SBMC was 0.52%; the average recovery rate for SBMO 0.65% (FIG. 4). In addition, of the three feed matrices evaluated, the least variation of EhV-86 content from the mean was in CF (2.10×10³ and 2.82×10³, 1^{st} and 3^{rd} quartiles, respectively). There was also greater EhV-86 deviation from the mean in SBMC than in SBMO (FIG. 4). These results suggested that sampling sensitivity was highest in CF followed by SBMO and lastly SBMC. This finding was noted when evaluating results to determine the most effective virus inactivation methods.

After the 23-day commercial trucking journey across the United States, an average of 6.81×10³ EhV-86/μL (Ct 22.2), or 3.41×10⁵ EhV-86/g, was recovered from the CF matrix. This represented an average recovery rate of 0.52%, with a 289% increase in virus concentration compared to day 0 (FIG. 4). Similarly, an average of 1.87×10³ EhV-86/μL (Ct 25.3), or 9.37×10⁴ EhV-86/g, was detected in SBMC (0.14% recovery or 28% more viruses) on day 23. For SMBO, an average of 6.25×10³ EhV-86/μL (Ct 24.0), or 3.13×10⁵ EhV-86/g, was recovered, which equates to 0.47% recovery and a 73% increase in virus concentration compared to EhV-86 concentrations on day 0 (FIG. 4). These results indicated that there was greater variation of virus quantity recovered in the SBM matrices compared with CF, meaning that sampling sensitivity was highest in CF followed by the SBMO and SBMC matrices.

### Viable Virus Detected on Feed Matrices Transported Across the United States

On day 0, an average of 2.19×10² EhV-86/μL (Ct 29.0), or 1.10×10⁴ EhV-86/g, viable virus was detected with the V-qPCR assay from the CF matrix. This represented an average of 0.02% recovery rate and 9% viability at the start of the study when compared with the eluted virus counts on day 0. On day 23, an average of 7.46×10² EhV-86/μL (Ct 25.5) or 3.73×10⁴ EhV-86/g was recovered representing an average rate of 0.06%. This resulted in a range from 11% to 32% in viability depending on whether the day 23 or day 0 standard qPCR results were used as baselines, respectively. These results represented an increase in viability of 2% to 22%, or no loss in viability due to the 23-d transport event.

Similarly, an average of 2.08×10³ EhV-86/μL (Ct 25.1), or 1.04×10⁵ EhV-86/g, viable virus was detected on day 0 from SBMC. This represented an average 0.16% recovery rate and 31% viability at the beginning of the study. On day 23, 9.10×10² EhV-86/μL (Ct 26.2), or 4.55×10⁴ EhV-86/g, was measured, which equates to 0.07% recovery rate and a range in viability from 13% to 49%, depending on whether the day 0 or day 23 standard qPCR results were used as baselines, respectively (FIG. 4). This represents a range from a 17% loss in viability to a gain of 18% in viability compared to standard qPCR (herein referred to as “S-qPCR”) results. Given the large deviation from the mean for the first and third quartiles observed for both the standard and viability qPCR data (FIG. 4), no significant loss of virus was also observed in SBMC matrix over the 23-d transport period. It was learned that the sub sampling method should be taken into consideration when evaluating the result of this assay.

Finally, an average of 4.52×10² EhV-86/μL (Ct 27.7), or 2.26×10² EhV-86/g, viable virus was detected on day 0 from SBMO. This represented an average 0.003% recovery rate and 0.05% viability at the start of the study. On day 23, 8.31×10² EhV-86/μL (Ct 27.1), or 4.15×10⁴ EhV-86/g, was detected, which equates to a 0.06% recovery rate and a 10% to 13% range in viability depending on whether the day 0 or day 23 S-qPCR results were used as baselines, respectively. These results represented a gain of 10% to 13% in viability compared to S-qPCR results. As for the SBMC matrix, the large deviation from the mean, or 1^{st} and 3^{rd} quartiles observed for both the standard and viability qPCR data (FIG. 4), indicated that no significant loss of virus occurred in the SBMO matrix over the 23-day transport period.

### Variation in EhV-86 Quantity Across the Matrices

There was a large range in EhV-86 quantity in the SBM matrices compared with the CF matrix (FIG. 4). The average virus quantity for CF (S-qPCR) was much greater than in SBMC (for both S-and V-qPCR methods), SBMO (based on V-qPCR), and CF (based on V-qPCR) (p=0.006, 0.001, 0.002, and 0.001, respectively). The uninoculated negative control matrices had negative PCR results (no amplification observed after 40 cycles, data not shown) as expected.

### No PCR Bias was Observed to be Associated with the Matrix Type

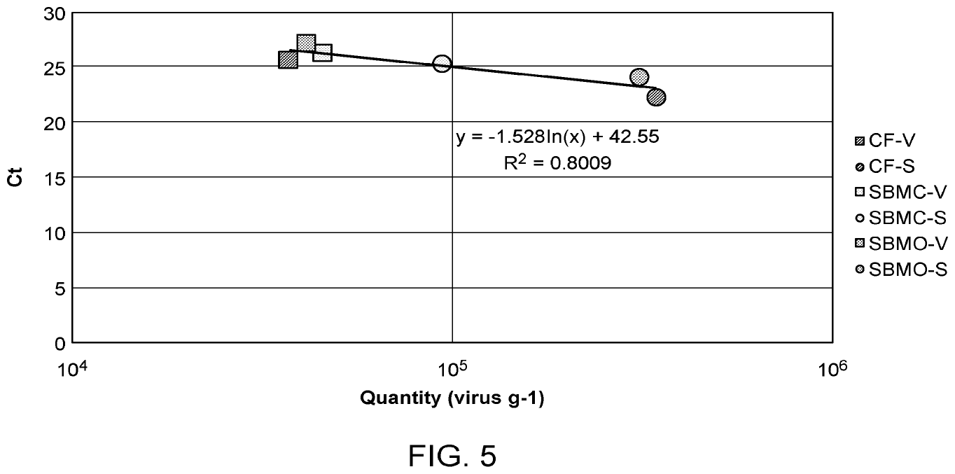

A standard curve (y=−1.528ln(x)+42.55) was created by plotting virus quantity (virus/g) against Ct values in the various feed matrices (both V-and S-qPCR) (FIG. 5). The standard curve had an R² value of 0.8009 which suggested that the type of matrix did not confound the qPCR assay and could be used to accurately quantify amounts of virus for all of these feed matrices. The EhV-86 qPCR assay in feed matrices had a quantifiable range of 6,800 to 340,567 EhV-86 genomes per reaction (FIG. 5).

Thus, this disclosure describes an exemplary use of the algal NCLDV EhV-86 as a surrogate for ASFV to study virus survival in exemplary animal feed matrices subjected to environmental conditions during a 23-day trans-continental commercial trucking journey across the United States. While the conditions corresponded to trans-continental commercial trucking transportation, the data reported herein are also relevant to storage conditions without transportation. Overall, viral load (virus/μL) in samples was successfully quantified using the novel technology of the V-qPCR assay.

Current conventional methods used to measure ASFV inactivation include hemadsorption (HAD₅₀) tests, plaque assays, an EGFP-fluorescent technique, swine bioassays, median tissue culture infectious dose (TCID₅₀) or cytopathic effect (CPE) assays, real-time RT-PCR, and quantitative PCR (qPCR). Although the quantification of the amount of virus nucleic acids using the qPCR method is useful, it fails to distinguish nucleic acids from viable versus non-viable viruses. Viability PCR (V-PCR or V-qPCR) uses viability dyes, such as ethidium monoazide (EMA) or propidium monoazide (PMA), prior to nucleic acid extraction and PCR or RT-PCR evaluation to evaluate infectivity of a virus. Propidium monoazide (PMA) is a photoreactive, membrane-impermeant dye that will selectively penetrate cells that have compromised cell membranes, and thereby considered dead. These dyes will bind to the nucleic acids, which then inhibits this DNA from amplifying during PCR amplification. Viability reverse transcriptase-qPCR can be used to evaluate the viability of Porcine epidemic diarrhea virus (PEDV) exposed to heat treatments so that viability reverse transcriptase-qPCR may be used to monitor PEDV contamination in feed and feed ingredients.

In this transcontinental United States transport scenario, EhV-86 viral DNA was present in the complete feed (average Ct value of 22.2 or 3.4×10⁵ virus/g), conventional soybean meal (average Ct value of 25.3 or 9.37×10⁴ EhV-86/g), and organic soybean meal (average Ct value of 24.0 or 3.13×10⁵ EhV-86/g) after 23 days of transport, which implied that NCLDVs such as ASFV are relatively stable in certain feed matrices. In addition to detecting viral genome in these three types of feed matrices, viable EhV-86 was quantified in complete feed (average Ct value of 25.5 or 3.13×10⁵ EhV-86/g), conventional soybean meal (average Ct value of 26.2 or 4.55×10⁴ EhV-86/g), and organic soybean meal (average Ct value of 27.1 or 4.15×10⁴ EhV-86/g) after the 23-day transport period via viability qPCR. This provided empirical data that the NCLDV ASFV-like EhV-86 could remain viable in these exemplary swine feed matrices for more than three weeks.

Among the three different exemplary feed matrices, the variation in the quantity of standard and viable DNA was greatest in the organic soybean meal and least in the complete feed. An increase in the amount of virus was detected in complete feed after 23 days of transport compared with the amount on day 0 in standard qPCR analysis, and an increase in the amount of viable virus detected in complete feed and organic soybean meal in V-qPCR analysis. The factors contributing to these differences between feed matrices is unclear, but likely involve differences in their complexity and physiochemical properties (i.e., complex mixture of ingredients in complete feed compared with a single ingredient of soybean meal). Moisture content and water activity of feed matrices may play a role in survival of some viruses in some feed matrices, but no studies have been conducted to evaluate this possibility with NCLDVs. Understanding the amount and variation of viable virus in feed matrices is important because ASFV is extremely resilient and remains viable in a variety of environments and porcine tissues for many months. The minimum infectious dose (10⁴ TCID₅₀) of ASFV as determined can be transmitted orally through contaminated feed, and repeated exposures exponentially increase the likelihood of infection. This emphasizes the significance of the results reported herein regarding recovery of high concentrations of viable virus in these feed matrices under the environmental conditions of a 23-day transcontinental United States transport time period.

A minimum of 1,500 infectious EhV-86 per gram (g) of organic soybean meal was detected at the end of a 23-day transport period, which means that a pig consuming 1.4 kg of feed a day would be exposed to 9.26×10⁵ EhV-86 daily. If these values are representative for ASFV, then an ASFV infection would most likely be observed. However, it should be noted that these calculations are based on the methodological bias of EhV-86 being eluted from the feed. One must consider that >80% of the NCDLV remained in the feed matrix, either in a potentially viable or degraded form, since only about 20% of the added virus was eluted from the whole SBMC sample. Thus, the remaining 80% was not in the eluent, thereby remaining associated to the matrix in a viable or degraded form.

Currently, chemical mitigation strategies involving formaldehyde, medium chain fatty acids, and glycerol monolaurate have been shown to reduce ASFV infectivity in feed matrices in laboratory settings. These mitigation strategies need to be evaluated in full scale commercial feed mills and supply chains, which may be possible by using a suitable surrogate for ASFV such as EhV-86.

Thus, the NCLDV EhV-86 can be used as a surrogate for ASFV under real-world conditions. EhV-86 can be detected in a viable form collected from an experimentally inoculated animal feed matrix after a 23-day transcontinental truck transport journey. Additionally, sampling sensitivity, rather than virus degradation, likely explains the variation of in EhV-86 quantity detected after a 23-day transport period. These results demonstrate that ASFV-like NCLDVs can retain viability in swine feed matrices during long-term transport across the continental United States, thereby providing evidence for the use of EhV as a surrogate for ASFV in real-world demonstrations.

Thus, as indicated above, the surrogate virus can be used as a surveillance assay, providing real-world in situ data on the effectiveness of treatments in reducing a megavirus from complete feed even after extended periods of transport or storage. The data provided herein establish that the surrogate virus can remain viable under real-world commercial transportation and/or storage conditions, making the assays described herein directly relevant to assessing the safety of animal feed treated to reduce the presence of megavirus from the feed, even after real-world commercial transportation and/or storage conditions.

Suitable treatments to reduce the presence of megavirus in animal feed or an animal feed ingredient include, but are not limited to treatment with citric acid, adding salt to the feed or feed ingredient, or other physical or chemical treatment. For example, citric acid (2%) is recommended as a disinfectant for ASFV-contaminated surfaces. As much as 10⁶ CCID₅₀/mL of virus, when applied to wood surfaces, were completely inactivated after 30 minutes of washing the surface with 2% citric acid. Citric acid is also an acceptable feed ingredient for use in swine feeds. Adding 0.05% salt to a corn-soybean meal-based diet inactivated 1-log of PEDV within 11.4 days compared to 17.2 days in the same diet without salt. Likewise, survival of swine viruses was less in complete feed than in individual feed ingredients. These observations suggest that increased salinity may modify properties of feed that decrease virus survival.

The surrogate virus assay can therefore be used to measure the effect of temperature, citric acid, salinity, and/or other physical or chemical treatment on the inactivation kinetics of a megavirus, such as, for example, ASFV and/an ASFV-like surrogate megavirus (e.g., EhV) in feed or in a feed ingredient. The animal feed or animal feed ingredient can be assayed prior to transportation or storage. If megavirus is detected, the animal feed or animal feed ingredient may be spike with surrogate virus, then treated to reduce megavirus and surrogate virus in the animal feed or animal feed ingredient. After transportation or storage, assaying the animal feed or animal feed ingredient for presence of the surrogate virus indicates the likelihood and extent to which megavirus may be present within the animal feed or animal feed ingredient.

Thus, the assay performed after transportation or storage may occur at any appropriate time after the animal feed or animal feed ingredient is loaded for transportation or storage, regardless of whether the animal feed or animal feed ingredient is transported. In one or more embodiments, the assay performed after transportation or storage may occur a minimum of at least 14 days such as, for example, at least 20 days, at least 21 days, at least 23 days, at least 24 days, at least 25 days, at least 26 days, at least 27 days, at least 28 days, at least 29 days, at least 30 days, at least 60 days, at least ninety days, at least 120 days, at least 150 days or at least 180 days after the animal feed or animal feed ingredient is spiked with surrogate virus, treated to reduce megavirus and the surrogate virus, then subjected to real-world conditions.

In one or more embodiments, the assay performed after transportation or storage may occur a maximum of no more than five years, no more than two years, no more than one year, no more than six months, no more than two months, or no more than one month after the animal feed or animal feed ingredient is spiked with surrogate virus, treated to reduce megavirus and the surrogate virus, then subjected to real-world conditions.

In one or more embodiments, the assay performed after transportation or storage may occur at a time within a range having as endpoints of any minimum period of time listed above and any maximum period of time listed above that is greater than the minimum period of time. For example, the predetermined period of time may be from 20 days to two months, from 60 days to five years, from 23 days to six months, etc., after the animal feed or animal feed ingredient is spiked with surrogate virus, treated to reduce megavirus and the surrogate virus, then subjected to real-world conditions.

In one or more embodiments, the assay performed after transportation or storage may occur at a time equal to any minimum time or maximum time identified above. Thus, the assay performed after transportation or storage may occur at, for example, 20 days, 21 days, 22 days, 23 days, 24 days, 25 days, 30 days, six months, two years, or five years after the animal feed or animal feed ingredient is spiked with surrogate virus, treated to reduce megavirus and the surrogate virus, then subjected to real-world conditions.

The assay can be performed on animal feed or an animal feed ingredient that has been subjected to conditions that include transportation or storage at temperatures up to and including 100° C., including, for example, conditions that include transportation or storage at a temperature of up to and including 90° C., up to and including 80° C., up to and including 70° C., or up to and including 60° C.

Again, while described herein in the context of an exemplary embodiment in which the Coccolithovirus EhV-86 is used as a surrogate for the Asfivirus ASFV, the assays described herein can be performed using any suitable surrogate for an infectious virus. The following discussion of structural characteristics and functional evaluation of EhV-86 infection potential is instructive of the structural and functional characteristics one can consider to determine whether a candidate surrogate virus is an appropriate surrogate for an infectious virus to be surveilled.

The virion architecture of ASFV includes two icosahedral protein capsids and two lipoprotein membranes. The outer lipoprotein membrane of ASFV is acquired at the end of the infection as the viral particles bud from the plasma membrane. The phycodnavirus nucleocapsids such as that of the chloroviruses also have icosahedral symmetry and the diameter of 130-200 nm. The only exception in nucleocapsid structure within the phycodnaviruses are the coccolithoviruses, including EhV. These virions also acquire an outer envelope via budding similarly to animal viruses such as ASFV viruses (Mackinder et al., 2009, J Gen Virol 90:2306-2316).

Unlike AFSV, however, the outer membrane of EhV-86 tightly adheres to the capsid and is covered by another not previously described layer, which may be formed by membrane-anchored envelope proteins. The origin of this outer membrane-anchored envelope was previously proposed to be a result from budding through the host cell cytoplasmic membrane. The outer membrane-anchored envelope can, however, become detached from the EhV-86 virion resulting in decreased infectivity of the virus. This also occurs with ASFV, where the outer envelope is not required for successful infection because ASFV can be internalised via both constitutive receptor-independent macropinocytosis and clathrin-mediated endocytosis.

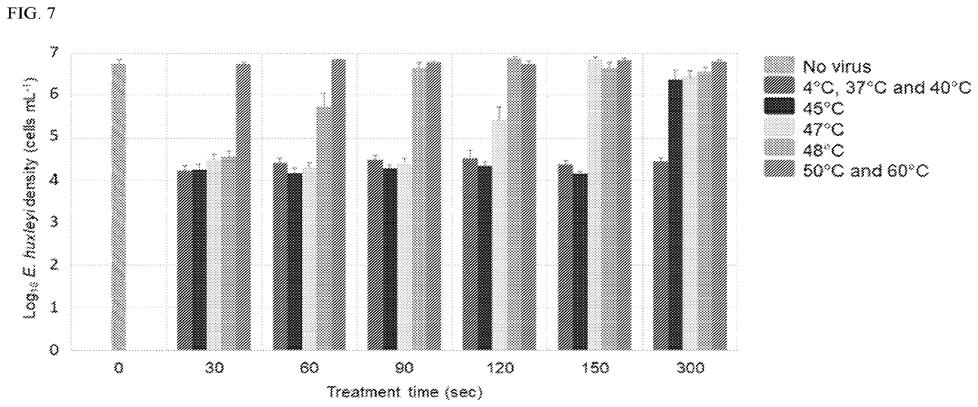

To evaluate whether the complex structure contributes to the thermal tolerance of EhV, EhV was treated with various time and temperature exposures. In field studies, EhV is genetically stable. Here, EhV-86 (0.8×10⁸ EhV particles as measured by flow cytometry) was treated at temperatures ranging from 4° C. to 60° C. for intervals of 30 seconds to five minutes, affirming routine lab observations that EhV-86 remained infectious at 4° C., 37° C., and 45° C. (FIG. 7). Bioassay infectivity conditions were defined as at least 2.5-log (corresponding to 25-fold) reduction of an E. huxleyi cell culture density by an EhV-86 by inoculum, eight days post-infection (FIG. 7).

As temperature reached 45° C. and above, there was a decrease on EhV-86 infectivity (FIG. 7). Thermal treatments at 47° C. and 48° C. resulted in reduced infectivity after 150 seconds and 90 seconds, resulting in an average reduction of host cell densities of 1.3 log and 1 log, respectively. Temperatures of 50° C. and 60° C. were sufficient to abolish viral infection after 30 seconds (FIG. 7).

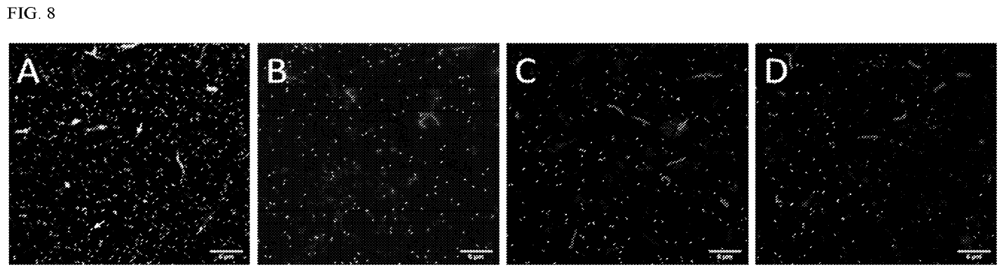

Confocal microscopy was used to confirm the structural integrity of the EhV virion after temperature and time treatments, (i.e., virus DNA can only be visualised if it is constrained by the virus membrane and/or capsid; FIG. 8). On average, 515 (SD=44) particles per image (n=3) for the EhV-86 exposed to 4° C., which corresponds to a virus concentration of 2.6×10⁸ EhV/mL (FIG. 8A), a concentration similar to the value obtained by flow cytometry. After a 20-minute treatment at 60° C., 80° C., or 100° C., the average number of DNA-containing particles recorded per image was 191 (SD=20), 195 (SD=44) and 180 (SD=44), respectively, corresponding to 0.43 log reduction in the number of viral particles relative to the 4° C. treatment (FIG. 8B-D). Presence of genome-containing particles at 60° C., 80° C., and 100° C. was unexpected as the bioassays indicated a loss in virus infectivity in treatments of 45° C. and above (FIG. 7).

The mean ratio of fluorescence signals of DNA (as stained by DAPI) and lipid membrane (as stained by FM 1-43 dye) of EhV-86 particles at 4° C. is 0.54. This ratio increased with increasing temperature to 1.19 at 60° C. and peaked at a ratio of 3.54 at 80° C. The ratio was reduced to 1.58 at 100° C. Similar to the DNA-to-lipid membrane signal ratio, the average DNA signal per virus particle (DAPI) also increased with increasing temperature from 153 Relative Fluorescence Units (RFU) at 4° C. to 351 at 60° C. and 433 at 80° C. The DNA signal was reduced to 242 RFU at 100° C. but remained higher than that observed at 4° C. The lipid membrane signal (FM 1-43) was similar at both 4° C. and 60° C., measuring at 301 and 306 RFU, respectively. The signal decreased at 80° C. and 100° C. to 128 and 161 RFU, respectively. These results suggest that the treatment at 60° C. resulted in most of the virions being damaged with the damage being the loss of the outer membrane. Given that DAPI passes through viable cell membranes less efficiently compared to damaged cells, the loss of outer membrane may have resulted in the DAPI stain penetrating the virion more efficiently resulting in the higher DNA signal at 60° C. versus 4° C. These results suggest that the loss of the infectious state of the virus population (even at temperatures greater than 45° C.) is not a function of the loss of virus genomes from the particles but rather the damage to the outer membrane and the associated envelope complex. Increasing the temperature to 80° C. or 100° C. resulted in the loss of this outer membrane (loss in FM 1-43 signal). Most of the damaged particles had either a denatured capsid plus inner membrane or only the internal membrane to surround their genomes. This is supported by the increased DNA labelling efficiency at 80° C. A loss in the DNA signal relative to 60° C. and 80° C. treatments were only observed when treating at 100° C., indicating the likely beginning of the virus DNA degradation.

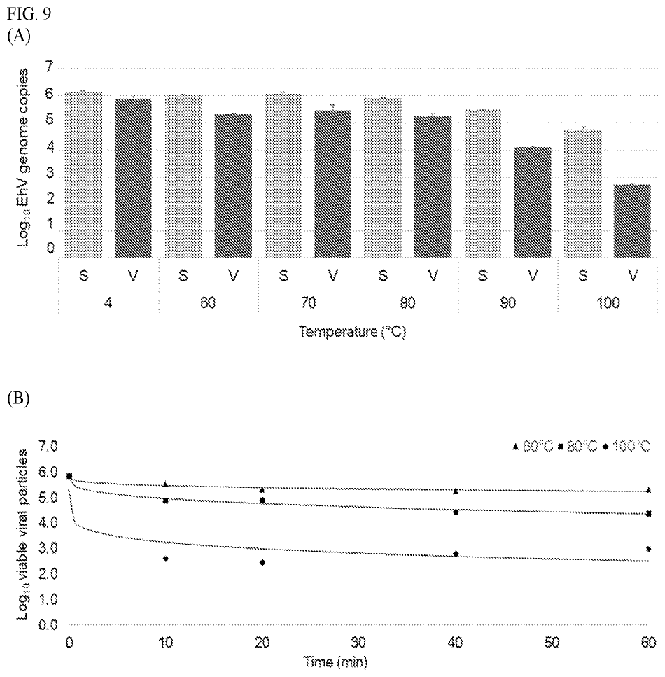

To further characterize this effect that high temperatures have on EhV-86 particles, V-qPCR was used to verify the microscopy observation. Even when EhV-86 was exposed to temperatures up to 80° C. for up to 20 minutes, less than 1-log reduction in the number of total virus genomes occurred (FIG. 9A). Temperatures greater than 80° C. resulted in reduction of the number of genomes by an additional 1 log, with 100° C. resulting in around 3.5 log reduction (FIG. 9A). The Weibull model (a continuous probability distribution model applied over exposure time at different temperatures) was applied to the V-qPCR results because of the nonlinear or tailing phenomena observed in the data (FIG. 9B). The delta values (thermal resistance or time at certain temperatures to reduce 1 log of the initial starting virus numbers) for EhV-86 were 410.5 minutes, 14.5 minutes, and 0.02 minutes at 60° C., 80° C., and 100° C., respectively. The time required to reduce EhV-86 infectivity by 3 log was 541.7 hours, 11.6 hours, and 0.4 hours at 60° C., 80° C., and 100° C., respectively.

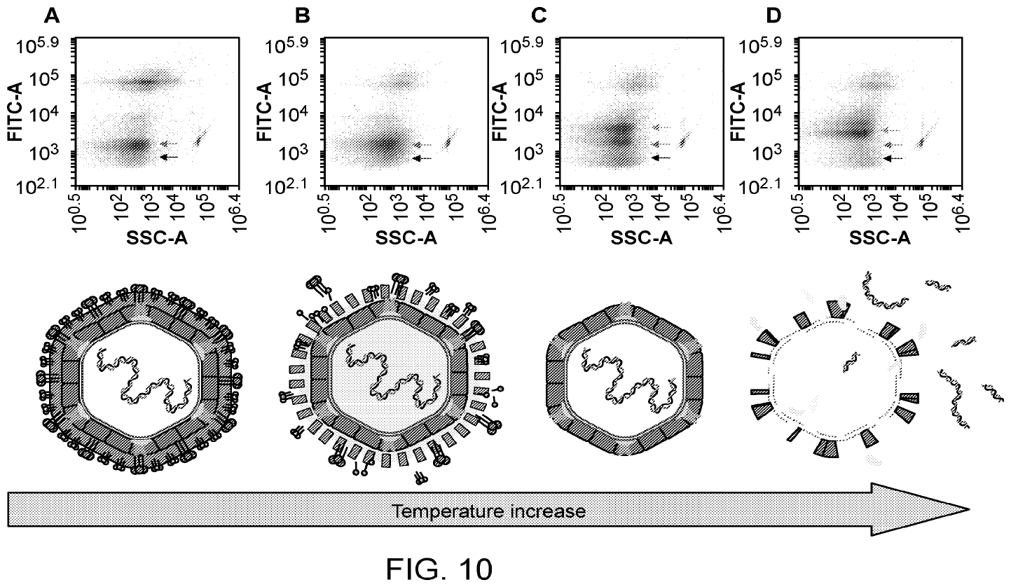

Flow cytometry was used to evaluate the effect of temperature on the ultra-structure degradation of EhV-86. Flow cytometry revealed that the SYBR nucleic acid staining efficiency of EhV-86 increased with increasing temperatures (FIG. 10). No discernible changes were observed in the particle properties as measured by side-scatter (SSC), which measures the light scatter at a ninety-degree angle relative to the laser (FIG. 10A). Even at 4° C., the population of EhV-86 particles was not homogeneous (black & blue arrows, FIG. 10A). When comparing flow cytometry plots of 4° C. and 48° C., a discernible increase in fluorescence in the EhV-86 population was observed (FIG. 10A-B). At 60° C. and 80° C., many more destroyed virions were observed (red arrows, FIG. 10C-D), with all particles showing evidence of some stage of degradation. In general, the extent of the degradation increased with increasing temperatures.

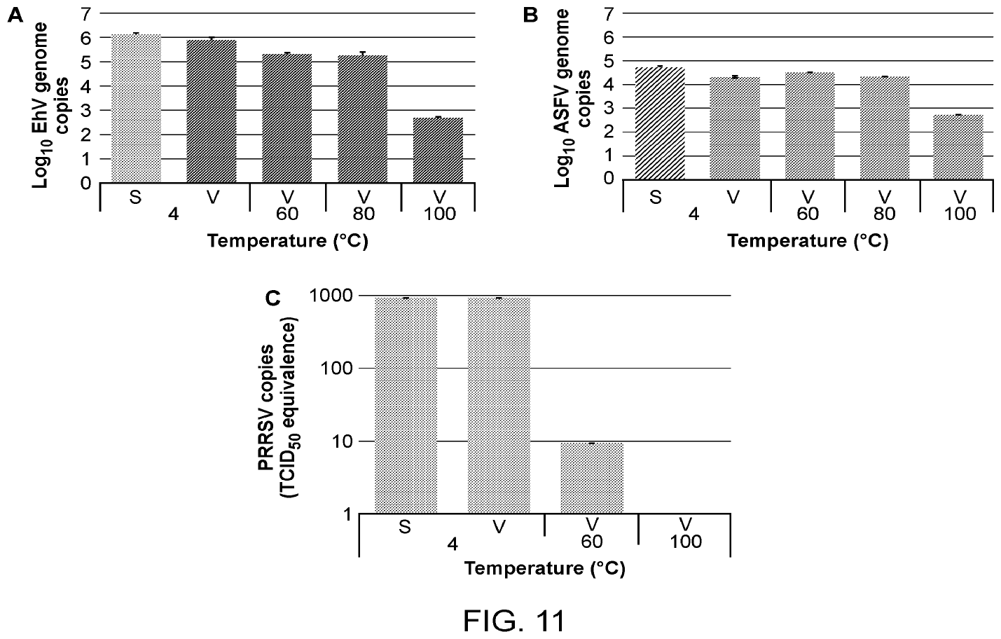

Finally, heat inactivation of ASFV and EhV were compared. Both viruses were similarly negatively affected by heat (FIG. 11A-B), i.e., both showed thermal tolerance up to 80° C. (<1-log reduction in viable virus copies). Both were sensitive to 100° C. treatment, showing between 2-and 3-log reduction in viable viruses. In contrast, the single positive-stranded RNA virus PRRSV was shown to be unstable at 60° C. and was not detected at 100° C. (FIG. 11C).

In summary, megaviruses such as EhV-86 and ASFV retain their genomes even after exposure to temperatures greater than 80° C., while RNA viruses such as PRRSV do not. Given this, there is a possibility that particles with a disrupted outer membrane but with intact capsid, inner membrane, and genome could potentially infect E. huxleyi and swine macrophages if acquired by phagocytosis or similar endolytic event. Based on the observed stability of genomes inside the damaged particles of EhV-86 or ASFV and their potential role in infection, both may be able to form a viable but non-infectious particle (VNIP). The VNIP concept and its potential role in causing disease may be analogous to the viable but non-culturable state observed for enteric bacteria in the marine environment. This is of particular importance when one considers that lytic viruses that infect unicellular organism spend most of their life span outside hosts.

In the preceding description and following claims, the term “and/or” means one or all of the listed elements or a combination of any two or more of the listed elements; the terms “comprises,” “comprising,” and variations thereof are to be construed as open ended-i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.). The term “in the range” or “within a range” (and similar statements) includes the endpoints of the stated range.

In the preceding description, particular embodiments may be described in isolation for clarity. Reference throughout this specification to “one embodiment,” “one or more embodiments,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXEMPLARY EMBODIMENTS

Embodiment 1. A method for monitoring the viability of a megavirus in an animal feed or an animal feed ingredient, the method comprising:
inoculating the animal feed or animal feed ingredient with a surrogate virus as a proxy for the megavirus;
subjecting the animal feed or animal feed ingredient to a treatment that inactivates the megavirus and the surrogate virus;
subjecting the animal feed or animal feed ingredient to storage or transportation conditions for at least 14 days; and
determining the viability of the surrogate virus in the animal feed or animal feed ingredient, thereby monitoring the viability of the megavirus in the animal feed or animal feed ingredient.

Embodiment 2. The method of embodiment 1, wherein the megavirus is African swine fever virus (ASFV) and the surrogate virus is an ASFV surrogate virus.

Embodiment 3. The method of embodiment 2, wherein the ASFV surrogate virus comprises a Coccolithovirus.

Embodiment 4. The method of embodiment 3, wherein the Coccolithovirus is Emiliania huxleyi virus.

Embodiment 5. The method of embodiment 2, wherein the treatment that inactivates infectious ASFV and the ASFV surrogate virus comprises exposure to a temperature of at least 65° C. for at least one minute, exposure to a temperature of at least 85° C. for at least one second, exposure to citric acid, or exposure to increased salinity.

Embodiment 6. The method of embodiment 1, wherein the animal feed or animal feed ingredient is subjected to storage or transportation conditions for at least 120 days.

Embodiment 7. The method of embodiment 6, wherein the animal feed or animal feed ingredient is subjected to storage or transportation conditions for at least 180 days.

Embodiment 8. The method of embodiment 1, wherein the conditions of storage or transportation include temperatures up to and including 100° C.

Embodiment 9. The method of embodiment 1, further comprising determining that the animal feed or animal feed ingredient with no detectable viable surrogate virus is safe for livestock.

Embodiment 10. The method of embodiment 1, further comprising identifying a treatment that results in no detectable viable surrogate virus as effective to permanently inactivate the megavirus.

Embodiment 11. The method of embodiment 1, wherein determining the viability of the surrogate virus comprises performing viability PCR or viability qPCR and interpreting the results.

Embodiment 12. The method of embodiment 1, further comprising determining the viability of a virus unrelated to either the surrogate virus or the megavirus.

Embodiment 13. The method of embodiment 12, wherein the virus unrelated to either the surrogate virus or the megavirus is Porcine reproductive and respiratory syndrome virus.

Embodiment 14. A method for monitoring the viability of a megavirus in an animal feed or an animal feed ingredient, the method comprising:
inoculating the animal feed or the animal feed ingredient with a surrogate virus as a proxy for the megavirus;
subjecting the animal feed or the animal feed ingredient to a treatment that inactivates the megavirus and the surrogate virus;
subjecting the animal feed or the animal feed ingredient to storage or transportation conditions for at least 14 days; and
determining the viability of the surrogate virus in the animal feed or the animal feed ingredient, thereby monitoring the viability of the megavirus in the animal feed or the animal feed ingredient.

Embodiment 15. The method of embodiment 14, wherein the animal product is subjected to storage or transportation conditions for at least 120 days.

Embodiment 16. The method of embodiment 15, wherein the animal feed or animal feed ingredient is subjected to storage or transportation conditions for at least 180 days.

Embodiment 17. The method of embodiment 14, wherein the conditions of storage or transportation include temperatures up to and including 100° C.

Embodiment 18. The method of embodiment 14, wherein determining the viability of the surrogate virus comprises performing viability PCR or viability qPCR and interpreting the results.

Embodiment 19. The method of embodiment 14, further comprising determining the viability of a virus unrelated to either the surrogate virus or the megavirus.

Embodiment 20. The method of embodiment 19, wherein the virus unrelated to either the surrogate virus or the megavirus is Porcine reproductive and respiratory syndrome virus.

The present invention is illustrated by the preceding exemplary embodiments and the following examples. It is to be understood that the particular examples, materials, amounts, and procedures are to be interpreted broadly in accordance with the scope and spirit of the invention as set forth herein.

### EXAMPLES

### Example 1

### Cell Culture and EhV-86 Stock

A culture of Emiliania huxleyi CCMP374, courtesy of Dr. Martinez-Martinez laboratory (Bigelow—Laboratory for Ocean Sciences, Maine) was grown in ALGA-GRO Seawater Medium (Carolina Biological Supplement Co., Burlington, NC) at 15° C. with 18-hour/6-hour light/dark cycle (ca. 2400 lux) until the concentration of 2×10⁵ cells/mL was reached. Isolate EhV-86, also courtesy of Dr. Martinez-Martinez laboratory, was added to E. huxleyi at a multiplicity of infection (MOI) of 1 and grown in a 15° C. incubator until lysis was observed, which was usually after four days. The lysate was filtered through a 0.45 micrometer (μm) filter (NALGENE RAPID-FLOW Bottle Top Filters, Thermo Fisher Scientific, Inc., Waltham, MA) to remove cell debris. This filtration and infection procedure was repeated several times. The filtered lysate was divided into aliquots and kept in the dark at 4° C. until use.

### Feed Matrices

Feed ingredients included conventional soybean meal (SBMC: containing 1-2% oil and 46-47% crude protein), organic soybean meal (SBMO: containing 6-7% oil and 44-45% crude protein), and a complete grower-finisher swine feed (CF: corn and soybean meal-based). For each feed matrix, four subsamples (30 g each) were weighed and placed into individual 50 mL mini-bioreactor tubes with vented caps. A total of three allotments per feed matrix were spiked with 2 mL EhV-86 (1×10⁹ viruses/mL) via injection using a 3 mL syringe with an 18-gauge needle. The remaining three samples served as negative controls with no virus added to the feed matrix.

### Transport Model

All feed samples were placed in a box on the trailer floor of a commercial semi-trailer truck with a 15.8-meter trailer (Csp Delivery, Fridley, MN). The truck carrying the inoculated feed samples departed from Minneapolis, Minnesota on Nov. 30, 2020, and returned on Dec. 22, 2020 (23 days). The route covered various regions of the United States including 29 states in the Midwest, Rocky Mountains, Southwest, Gulf Coast, Eastern Seaboard, New England region, and the Great Lakes region (FIG. 1). The temperature and humidity were recorded every 15 minutes during transport. The commercial truck did not encounter any unexpected stops or accidents. Upon completion of the journey, samples were removed from the truck and stored at −20° C. until analysis.

### Testing of Protocol for EhV-86 Elution From Soybean Meal

200 μL of EhV-86 filtrate (1×10⁹ cells/mL) was added to 1 g of SBMC in a 50 mL conical centrifuge tube (FALCON, Thermo Fisher Scientific, Inc., Waltham, MA), and held at room temperature for five minutes before eluting the virus from the matrix by adding 10 mL of ALGA-GRO Seawater Medium (Carolina Biological Supplement Co., Burlington, NC). The tube was vortexed repeatedly for one minute before incubating in a water bath (ISOTEMP 205 digital water bath, Thermo Fisher Scientific, Inc., Waltham MA) set at 40° C. for a total time of 30 minutes. The tube was removed and vortexed every five minutes for 30 seconds. At the end of the temperature exposure, the tube was centrifuged at 4700 rpm for five minutes to collect the matrix at the bottom of the tube. The supernatant (virus eluant) was removed and filtered through 0.22 μm syringe filter (MILLEX-GP sterile syringe filter with PES membrane, MilliporeSigma, Burlington, MA) into a centrifugal tube (AMICON Ultra 15 mL, MilliporeSigma, Burlington, MA). The virus eluant was repeatedly washed (three times) with 1× phosphate buffered saline (1×PBS, Thermo Fisher Scientific, Inc., Waltham, MA) via centrifugation as per manufacturers instruction and the final 200 μL volume obtained was split into two aliquots of 100 μL each. This was repeated to create biologically independent replicate samples.

qPCR and v-qPCR Assays

All assays were conducted in triplicate. To one set of 100 μL virus eluants previously mentioned, PMAxx dye (Biotium Inc., Fremont, CA, 25 micromolar (μM) final conc.) was added by following methods optimized as described in EXAMPLE 2 (Balesteri et al., 2022). This was the viability (V) set of treatments. An untreated duplicate set (i.e., no addition of PMAxx dye) of samples served as an untreated control template for standard(S) qPCR. All samples were incubated in the dark at room temperature for 10 min on a rocker to mix. The treated V samples were then exposed for 30 min to light using PMA-Lite device (Biotium Inc., Fremont, CA) to cross-link PMAxx dye to the DNA (free or within broken viruses). The duplicate S samples were kept in the dark at room temperature for the same length of time. All samples were then used for DNA extraction (QIAAMP MINELUTE virus spin, Qiagen, Hilden, Germany). The final 30 μL elution volumes were stored at 4° C. until qPCR analysis was conducted.

One μL from all the samples (virus eluants and DNA extractions) served as the DNA template in the subsequent 20 μL qPCR mix (QUANTINOVA SYBR Green PCR kit, Qiagen, Hilden, Germany): 10 μL Master Mix, 0.1 μL QN ROX Reference Dye, 1.4 μL reverse primer (GACCTTTAGGCCAGGGAG (SEQ ID NO:1); 0.7 μM final conc.), 1.4 μL forward primer (TTCGCGCTCGAGTCGATC (SEQ ID NO:2); 0.7 μM final conc), and 6.1 μL molecular grade water. The primers amplify part of the single copy major capsid protein (MCP) gene of EhV as previously described (Schroeder et al., 2003, Appl. Environ. Microbiol. 69:2484-2490). The qPCR analysis was conducted using a real-time PCR machine (QUANTSTUDIO 3, Thermo Fisher Scientific, Inc., Waltham, MA) run on the following qPCR conditions: two minutes at 95° C. followed by 40 cycles of five seconds at 95° C. and 10 seconds at 60° C.

Standards for the qPCR assays were created using EhV-86 as the template, with the MCP amplificon purity confirmed using E-Gel electrophoresis system (Thermo Fisher Scientific, Inc., Waltham, MA) and extracted using a gel DNA recovery kit (ZYMOCLEAN, Zymo Research, Irvine, CA). The number of EhV-86 genomic copies that equate to MCP copies in the extracted MCP amplicon product was calculated using the following formula:

$$No of copies = \frac{( ng \times 6.022 \times {10}^{23} )}{8345 4.93 Da \times 1 \times {10}^{9}}$$

where ng is the amount of the MCP amplicon as measured by Qubit4 (Invitrogen, Thermo Fisher Scientific, Inc., Waltham, MA), 6.022×10²³ is Avogadro's number, 83454.93 Da is the molecular weight of our MCP amplicon as calculated using the Sequence Manipulation Suite (Stothard, P., 2000, Biotechniques 28(6):1102-1104) and 1×10⁹ is used to convert the molecular weight of the amplicon to nanograms. A dilution series of the MCP amplicon was used to create a EhV-86 genomic equivalent standard curve. Fresh dilutions for the standard curve were made for every qPCR run.

### Protocol for EhV-86 Elution From Feed Matrices Used in Transport Study

One gram of each 30 g of feed matrix was sampled on day 23 and was used in the virus elution protocol as previously described. The qPCRs were carried out on DNA extracted from the eluant. The percentage of virus recovered was calculated as follows:

$$\frac{amount of virus per gram recovered after treatment and analysis}{starting amount of virus per gram} \times 100 \%$$

### Statistical Analysis

Visualization of data was performed using the ggplot2 package of RStudio environment (Version 1.1.456, RStudio, Inc., Boston, MA) using R programming language [Version 4.0.5 (2021-03-31), R Core Team, R Foundation for Statistical Computing, Vienna, Austria]. Viral quantity averages (virus/μL) were normally distributed and compared using two-sample t-test assuming unequal variances in Excel.

### Example 2

### Cell Culture and EhV-86 Stocks

A culture of Emiliania huxleyi CCMP374, courtesy of the Dr. Martinez-Martinez laboratory (Bigelow—Laboratory for Ocean Sciences, Maine, US) was grown in ALGA-GRO Seawater Medium (Carolina Biological Supplement Co., Burlington, NC) at 15° C. with an 18 hour/six-hour light/dark cycle (ca. 2400 lux) until the concentration of 2×10⁵ cells/mL was reached. Isolate EhV-86, also courtesy of Dr. Martinez-Martinez laboratory, was added to E. huxleyi at a multiplicity of infection (MOI) of 1 and grown in a 15° C. incubator until lysis was observed, which was usually after four days. The lysate was filtered through a 0.45 μm filter (NALGENE RAPID-FLOW Bottle Top Filters, Thermo Fisher Scientific, Inc., Waltham, MA) to remove cell debris. This filtration and infection procedure was repeated several times. The filtered lysate was divided into aliquots and kept in the dark at 4° C. until use.

Pathogenic ASFV strain Pretoriuskop/96/4 (Pr4), isolated from Ornithodoros porcinus porcinus ticks collected from the Republic of South Africa in 1996, was obtained from the Plum Island Animal Disease Center reference collection.

Virulent PRRSV L1A or 1-4-4 variant was maintained in MARC-145 cells were used for the isolation, propagation, and enumeration in preparation of the virus for subsequent stability assays (Schroeder et al., 2021, Microbiology Resource Announcements 10(33)).

### EhV-86 Bioassay

A culture of E. huxleyi CCMP347 was grown as previously described, and once the cells reached a density of 1×10⁶ cells/mL, the culture was divided into 900 μL aliquots and each aliquot was infected with 100 μL of EhV-86 (MOI of 1 based on flow cytometry), except for the uninfected negative controls. Virus treatments were conducted using 100 μL aliquots of EhV-86 (1×10⁴ EhV/μL) at 4° C., 37° C., 40° C., 42.5° C., 45° C., 47° C., 48° C., 50° C., 60° C., 80° C., or 100° C. in a thermocycler (MINIAMP Plus, Applied Biosystems, Thermo Fisher Scientific, Inc., Waltham, MA) for intervals of 30 seconds or 5 minutes. Each treatment at each time point had five replicates. Cell counts were determined on day 0 and day 8 post-infection (PI) using a hemacytometer (Neubauer improved, Marienfeld, Dortmund, Germany) and an optical microscope (Nikon Eclipse Ci, Nikon USA, Melville, NY). Cell number over time was plotted as the reduction in log cell number over time of treatment (in min or sec). The lower limit for counting of E. huxleyi cells in a hemacytometer was considered to be 1×10⁴ cells/mL.

### Confocal Microscopy

Four samples were prepared including one non-treated control EhV-86 kept at 4° C., and three EhV-86 samples treated for 20 minutes at 60° C., 80° C., or 100° C. For each sample, 500 μL of viral lysate were incubated with DAPI dilactate (Thermo Fisher Scientific, Inc., Waltham, MA) at a final conc. of 5 μg/mL overnight at 4° C. to stain the nucleic acids as previously described (Mackinder et al., 2009, J Gen Virol 90:2306-2316). Subsequently, FM 1-43 dye (Thermo Fisher Scientific, Inc., Waltham, MA) was added (10 μM final conc.), and solutions were incubated at 4° C. in the dark for 10 minutes to allow the staining of the lipid membranes. The stained viral lysate was filtered through 0.02 μm anodisc filter (WHATMAN ANOPORE, Sigma-Aldrich, St. Louis, MO), positioned on top of a disposable 0.45 μm filter apparatus previously wetted with 1 mL of dH₂O (pump pressure 100 mbar). After filtration, the filters were dried under a flow hood in the dark, until the filters appeared opaque. Each anodisc filter was mounted in water and topped with a #1.5 cover slip. The cover slips were sealed onto the slides with clear nail polish. Image acquisition was obtained using a Nikon A1Rsi Confocal (Nikon USA, Melville, NY) w/SIM Super Resolution with the following settings: objective Plan Apo Lambda 60× oil immersion, 1.4 NA, scan average 16, 2.2 micros/pixels, sequential acquisition, and 1.2 AU (28 μm). Three images were taken for each filter.

Every image was analysed with Fiji software (https://imagej.net/Fiji): pre-processed using ‘Subtract background, 50 pixels’ to both channels and segmented with DiAna plugin after application of ‘Median’ filter for the DAPI channel and ‘None’ for the FM 1-43 channel. The final co-localisation and fluorescence intensity analysis was obtained with DiAna plugin which utilised the mask of the DAPI channel to quantify the signal in the DAPI and FM 1-43 channels for each detected particle. We defined the average of the mean particle intensity derived by DiAna from the background corrected images as Relative Fluorescence Units (RFU).

Viability qPCR

Samples of virus filtrate from cells infected with EhV, or ASFV were exposed to 4° C., 60° C., 80° C., and 100° C. for 10 minutes, 20 minutes, 40 minutes, or 60 minutes. Samples of virus filtrate from cells infected with PRRSV were exposed to 4° C., 60° C., and 100° C. for 10 minutes, 20 minutes, 40 minutes, or 60 minutes. After treatment, the filtrate was split in two aliquots and 25 or 100 μM final conc. of PMAxx Dye (Biotium Inc., Fremont, CA) was added to one of the aliquots, and incubated in the dark at room temperature for 10 minutes on a rocker to mix. The mixed sample was then exposed for 30 minutes to the light, using PMA-Lite device (Biotium Inc., Fremont, CA), to cross-link PMAxx Dye to the DNA or RNA. QIAAMP MINELUTE virus spin (Qiagen, Hilden, Germany) was used to extract DNA and NUCLEOMAG Virus kit (Takara Bio, USA) was used to extract RNA from all the aliquots, and a qPCR analysis was finally conducted using QUANTINOVA SYBR Green PCR or RT-PCR kits (Qiagen, Hilden, Germany)

PCR conditions for EhV or ASFV were as followings: 2 minutes at 95° C. followed by 40 cycles of five seconds at 95° C. and 10 seconds at 60° C. (reaction mix components: 10 μL SYBR Green PCR Master Mix, 0.1 μμL QN ROX Reference Dye, 1.4 μL reverse primer (Table 1; 0.7 μM final concentration), 1.4 μL forward primer (Table 1; 0.7 μM final concentration), 6.1 μL molecular grade water, and 1 μL DNA template. Primer details can be found in TABLE 1

RT-qPCR conditions for PRRSV are as follows: 10 μL Master Mix, 0.2 μL RT mix, 0.5 μL reverse primer (0.25 μM final conc), 0.5 μL forward primer (0.25 μM final conc), 7.8 μL molecular grade water and 1 μL RNA template. The RT-qPCR analysis was conducted using a real-time PCR machine (ROTOGENE, Qiagen, Hilden, Germany) run on the following RT-qPCR conditions: 50° C. for 10 minutes, 10 minutes at 95° C. followed by 40 cycles of 15 seconds at 95° C. and 1 min at 60° C. Primer details can be found in TABLE 1

TABLE 1
Primers used in V-qPCR assays.

| Primer | Sequence | SEQ ID | Source |
|---|---|---|---|
| EhV R | GACCTTTAGGCCAGGGAG | NO: 1 | Schroeder et |
| EhV F | TTCGCGCTCGAGTCGATC | NO: 2 | al., 2003, |
| | | | Appl. Environ. |
| | | | Microbiol. |
| | | | 69:2484-2490 |
| ASFV R | ATGTCCAGATACGTTGCGTCCG | NO: 3 | |
| ASFV F | AGGAGGTATCGGTGGAGGGAAC | NO: 4 | |
| PRRSV R | CGCCCTAATTGAATAGGTGACTTAG | NO: 5 | |
| PRRSV F | TAAGTTAYACTGTGGAGTTYAG | NO: 6 | |

Standards for the qPCR assays were created using EhV-86 as the template, with the MCP amplificon purity confirmed using E-Gel electrophoresis system (Thermo Fisher Scientific, Inc., Waltham, MA) and extracted using a gel DNA recovery kit (ZYMOCLEAN, Zymo Research, Irvine, CA). The number of EhV genomic copies that equates to MCP copies in our extracted MCP amplicon product was calculated using the following formula:

$$No of copies = \frac{( ng \times 6.022 \times {10}^{23} )}{8345 4.93 Da \times 1 \times {10}^{9}}$$

where ng is the amount of the MCP amplicon as measured by Qubit4 (Invitrogen, Thermo Fisher Scientific, Inc., Waltham, MA), 6.022×10²³ is Avogadro's number, 83454.93 Da is the molecular weight of the MCP amplicon as calculated using the Sequence Manipulation Suite (Stothard, P., 2000, Biotechniques 28(6): 1102-1104) and 1×10⁹ is used to convert the molecular weight of the amplicon to nanograms. A dilution series of the MCP amplicon was used to create a EhV genomic equivalent standard curve. Fresh dilutions for the standard curve were made for every qPCR run. For ASFV, a 10-fold serial dilution of the ASFV p72 gene (synthetic construct from BioGX, https://www.biogx.com/) was used to create a standard curve to convert the Ct values from the RT-qPCR assay to ASFV genomic equivalents.

For PRRSV, a TCID₅₀ method, which is a routine method used for PRRSV quantification, was used to calculate the infectious unit of the PRRSV isolate. A RT-qPCR assay for PRRSV was designed to the GP5 structural protein and Ct values was converted to PRRSV TCID₅₀ equivalents taken from virus RNA extracted from a known titer.

### EhV Inactivation Kinetics

Experimental data on virus viable qPCR activity (log viable viral particles/mL) were plotted over exposure time (min) to obtain the survival curves. GinaFit software was used to estimate the kinetic parameters (delta-value, expressed as the time at certain temperature for the first-log viral decline) of the Weibull models (Mafart et al., 2002, Int J Food Microbiol 72:107-113). Goodness of fit of the model to the experimental data was expressed as the adjusted R²

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for monitoring the viability of a megavirus in an animal feed or an animal feed ingredient, the method comprising:
inoculating the animal feed or animal feed ingredient with a surrogate virus as a proxy for the megavirus;
subjecting the animal feed or animal feed ingredient to a treatment that inactivates the megavirus and the surrogate virus;
subjecting the animal feed or animal feed ingredient to storage or transportation conditions for at least 14 days; and
determining the viability of the surrogate virus in the animal feed or animal feed ingredient, thereby monitoring the viability of the megavirus in the animal feed or animal feed ingredient.

2. The method of claim 1, wherein the megavirus is African swine fever virus (ASFV) and the surrogate virus is an ASFV surrogate virus.

3. The method of claim 2, wherein the ASFV surrogate virus comprises a Coccolithovirus.

4. The method of claim 3, wherein the Coccolithovirus is Emiliania huxleyi virus.

5. The method of claim 2, wherein the treatment that inactivates infectious ASFV and the ASFV surrogate virus comprises exposure to a temperature of at least 65° C. for at least one minute, exposure to a temperature of at least 85° C. for at least one second, exposure to citric acid, or exposure to increased salinity.

6. The method of claim 1, wherein the animal feed or animal feed ingredient is subjected to storage or transportation conditions for at least 120 days.

7. The method of claim 6, wherein the animal feed or animal feed ingredient is subjected to storage or transportation conditions for at least 180 days.

8. The method of claim 1, wherein the conditions of storage or transportation include temperatures up to and including 100° C.

9. The method of claim 1, further comprising determining that the animal feed or animal feed ingredient with no detectable viable surrogate virus is safe for livestock.

10. The method of claim 1, further comprising identifying a treatment that results in no detectable viable surrogate virus as effective to permanently inactivate the megavirus.

11. The method of claim 1, wherein determining the viability of the surrogate virus comprises performing viability PCR or viability qPCR and interpreting the results.

12. The method of claim 1, further comprising determining the viability of a virus unrelated to either the surrogate virus or the megavirus.

13. The method of claim 12, wherein the virus unrelated to either the surrogate virus or the megavirus is Porcine reproductive and respiratory syndrome virus.

14. A method for monitoring the viability of a megavirus in an animal feed or an animal feed ingredient, the method comprising:
inoculating the animal feed or the animal feed ingredient with a surrogate virus as a proxy for the megavirus;
subjecting the animal feed or the animal feed ingredient to a treatment that inactivates the megavirus and the surrogate virus;
subjecting the animal feed or the animal feed ingredient to storage or transportation conditions for at least 14 days; and
determining the viability of the surrogate virus in the animal feed or the animal feed ingredient, thereby monitoring the viability of the megavirus in the animal feed or the animal feed ingredient.

15. The method of claim 14, wherein the animal product is subjected to storage or transportation conditions for at least 120 days.

16. The method of claim 15, wherein the animal feed or animal feed ingredient is subjected to storage or transportation conditions for at least 180 days.

17. The method of claim 14, wherein the conditions of storage or transportation include temperatures up to and including 100° C.

18. The method of claim 14, wherein determining the viability of the surrogate virus comprises performing viability PCR or viability qPCR and interpreting the results.

19. The method of claim 14, further comprising determining the viability of a virus unrelated to either the surrogate virus or the megavirus.

20. The method of claim 19, wherein the virus unrelated to either the surrogate virus or the megavirus is Porcine reproductive and respiratory syndrome virus.
